(19) United States
(12) Patent Application Publication
(10) Pub. No.: US 2018/0015421 A1
(43) Pub. Date: Jan. 18, 2018

(54) Pervaporation and Vapor-Permeation Separation of Gas-Liquid Mixtures and Liquid Mistures by Ion Exchanged SAPO-34 Molecular Sieve Membrane

(71) Applicants: Shanghai Advanced Research Institute, Chinese Academy of Sciences, Pudong, Shanghai (CN); Shanxi Lu'An Enviromental Protection & Energy Development Co., Ltd., Changzhi, SHX (CN); Total Raffinage Chimie, Courbevoie (FR)

(72) Inventors: Daniel CURULLA-FERRE, Uccle (BE); Yuhan SUN, Shanghai (CN); Zhiqiang SUN, Changzhi (CN); Yanfeng ZHANG, Shanghai (CN)

(21) Appl. No.: 15/547,933

(22) PCT Filed: Feb. 2, 2016
(86) PCT No.: PCT/EP2016/052175

§ 371 Date: Aug. 1, 2017

(30) Foreign Application Priority Data
Feb. 3, 2015 (CN) 201510054225.X

(51) Int. Cl. B01D 61/36; C07C 29/76; B01D 67/00; B01D 19/00; B01D 71/02; B01D 69/10; C07C 68/08; C01B 39/54

(52) U.S. Cl. CPC B01D 61/362; C07C 68/08; C07C 29/76; C01B 39/54; B01D 19/0031; B01D 71/028; B01D 69/105; B01D 67/0051; B01D 2323/46

(57) ABSTRACT

The invention discloses a method for the pervaporation and vapor-permeation separation of a gas-liquid mixture/liquid mixture by an ion-exchanged SAPO-34 molecular sieve membrane, said method comprises the following steps: 1) synthesis of SAPO-34 molecular sieve seeds; 2) coating the SAPO-34 molecular sieve seeds onto the inner surface of a porous support; 3) synthesis of SAPO-34 molecular sieve membrane; 4) performing ion exchange and calcination; 5) using the ion-exchanged SAPO-34 molecular sieve membrane obtained in step 4) to perform the separation of a gas-liquid mixture or a liquid mixture by a process of pervaporation separation or vapor-permeation separation. The present method for membrane separation of methanol-dimethyl carbonate has advantages like low energy consumption, being not limited by azeotropic mixture, high methanol flux and high separation factors and thus has great economic value.

### FIELD OF THE INVENTION

This invention relates to a method for separation of a mixture by a SAPO-34 molecular sieve membrane, especially a method for pervaporation (pervaporative separation) and vapor-permeation separation of a gas-liquid mixture or a liquid mixture by an ion-exchanged SAPO-34 molecular sieve membrane.

### BACKGROUND OF THE INVENTION

Dimethyl carbonate (DMC), which has a molecular formula of CO(OCH₃)₂, is a good solvent, has low volatility and similar toxicity values to anhydrous ethanol, and is completely biodegradable. It is an environmental-friendly chemical. Its molecules have an oxygen content of 53%, which is three times higher than that of methyl tert-butyl ether (MTBE). It can be used as an additive in gasoline to enhance octane number and to suppress emission of carbon monoxide and hydrocarbons. It is very active in terms of chemistry, and it is an important intermediate and starting material for organic synthesis. Dimethyl carbonate finds extensive applications in the fields of pharmaceutical, chemical engineering and energy etc, and is receiving increasing attention. It has been rapidly developed and is known as a new foundation of organic synthesis.

The industrial methods for producing DMC mainly include methods of oxidative carbonylation, transesterification, or phosgenation of methanol [Applied Catalysis A: General, 221(2001) 241-251]. No matter which method is used, a mixture of methanol (MeOH) and DMC was always obtained from the reaction. At normal pressure, MeOH and DMC would form a binary azeotrope (70 wt % MeOH and 30 wt % DMC), whose azeotropic temperature being 64° C. Therefore, it is a necessary to separate and recover DMC from the azeotrope. Currently, methods for separation of the MeOH/DMC azeotrope mainly include low temperature crystallization, adsorption, extractive distillation, azeotropic distillation and pressure distillation. All of these methods possess the disadvantages and shortcomings that energy consumption is high, it is difficult to select the appropriate solvent, it is difficult to operate and there are safety deficiencies. In contrast, a pervaporation method possesses advantages of low energy consumption, high efficiency and flexible operation conditions.

The pervaporation is a new membrane technology for separation. It uses the differential chemical potentials of a component on both sides of the membrane as a driving force. The membrane can be used to achieve selective separation of different components in feed liquids according to different affinity and mass transfer resistance of the components. Currently, the membranes used for pervaporation mainly include polymeric membrane, inorganic membrane and composite membrane. Recently, some progress has been made in studies on pervaporation separation of MeOH/DMC mixtures. Most of the studies focused on the polymeric membranes. The researchers found that materials such as polyvinyl alcohol (PVA), polyacrylic acid, chitosan or the like can be prepared into pervaporation membranes which preferentially remove methanol and have good separation performance.

Wooyoung et al. used a cross-linked chitosan membrane for pervaporation separation of MeOH/DMC and investigated the influences of operation temperature and feed composition on the separation factor and flux and received a good result [Separation and Purification Technology 31 (2003) 129-140]. Wang et al. prepared a polyacrylic acid (PAA)/polyvinyl alcohol (PVA) mixed membrane, wherein a mixed membrane containing 70 wt % PPA has a separation factor of 13 and a permeation flux of 577 g/(m² h) [Journal of Membrane Science 305 (2007) 238-246]. Pasternak et al. tested the performance of a polyvinyl alcohol (PVA) membrane for the separation of MeOH/DMC; for a feed composition of 70/30 MeOH/DMC, a methanol solution of 93˜97 wt% concentration is produced on the permeate side and the flux is 110-1130 g/(m² h) [U.S. Pat. No. 4,798,674 (1989)]. Chen et al. prepared a hybrid membrane of chitosan and silica through cross-linking chitosan with aminopropyl triethoxy silane. Separation factor of 30 and permeation flux of 1265 g/(m² h) were achieved at 50° C. for a 70/30 MeOH/DMC mixture.

The polymeric membranes have an advantage of low cost. However, they are also suffering from the disadvantages such as low chemical and thermal stability, easy to swell during the process of separation, and incapability of being used for separation at high pressure; all of which would influence the separation performance of the membranes. On the other hand, the inorganic membranes can well solve these issues because the inorganic membranes have a uniform pore size and high chemical and thermal stability. Therefore, the inorganic membranes can be used for separation in an environment under harsh conditions and they are also suitable for separation under high pressure. Currently, the main application of inorganic zeolite molecular sieve membranes is dehydration of organics. Applications of molecular sieve membrane in the separation of MeOH/DMC were rarely reported. Li et al. prepared a ZSM-5 molecular sieve membrane on porous alumina support and used the same for the separation of a water/acetic acid mixture [Journal of Membrane Science 218 (2003) 185-194]. Pina et al. synthesized a NaA molecular sieve membrane on Al₂O₃ support and used the NaA molecular sieve membrane to separate a water/ethanol mixture by pervaporation, in which the separation factor can reach 3600 and the permeation flux of water reaches 3800 g/(m² h) [Journal of Membrane Science 244 (2004) 141-150]. Hidetoshi et al. prepared NaX and NaY membranes on supports and systemically studied the pervaporation separation performance of the membranes. It was found that the membranes have very high selectivity to alcohols and benzene. They also studied the selectivity of these membranes for MeOH/DMC separation, and as a result, separation factor of 480 and permeation flux of 1530 g/(m² h) were achieved while the feed composition was 50/50 [Separation and Purification Technology 25 (2001) 261-268].

The separation performance of a molecular sieve membrane is influenced by many factors such as silicon/aluminum ratio of the framework, size of the seeds (crystal seeds), kinds of the template agent, thickness of the membrane, types of cations, properties of the support, calcining conditions, and defect-repairing method. Ion exchange is a simple but efficient method for improving the selectivity of a molecular sieve membrane.

Ion exchange of hydrogen ions in molecular sieve crystals for basic metal ions can enhance the basicity of the molecular sieve, and improve its absorption selectivity to acid gas (such as CO₂). Meanwhile, the incorporation of the metal ions will also change the channel size of the molecular sieve, thereby changing diffusion selectivity to gases. Walton et al. used various cations for ion exchange of X and Y molecular sieves, and the results indicated that the adsorption capacity of the molecular sieves exchanged with different ions increased in this order: Cs⁺<Rb⁺<K⁺<Na⁺<Li⁺[Micropor. Mesopor. Mater. 91(2006)78]. Yang et al performed ion exchange of high Si Beta molecular sieve with alkali metals and alkaline-earth metals and found adsorption capacity of the molecular sieves exchanged with different ions increased in this order: Mg²⁺<Cs⁺<Ca²⁺<Ba²⁺<Li⁺<Na⁺<K⁺[Micropor. Mesopor. Mater. 135(2010)90]. Kusakabeet et al. reported that the alkali metal ion exchanged NaY-type molecular sieve membrane has higher permeation rate than the alkaline earth metal ion exchanged NaY-type molecular sieve membrane [J. Membr. Sci. 148(1998)13]. Hasegawa et al. found that CO₂/N₂ separation selectivity of the NaY molecular sieve increased from 19 to 30˜40 after ion exchange with K⁺, Rb⁺and Cs⁺[Sep. Purif. Technol. 22-23 (2001) 319]. Jihong Sun et al. synthesized a lithium-type X molecular sieve having low-silicon and low-aluminum by firstly using a lithium ion aqueous solution to make a Na-type X molecular sieve having low-silicon and low-aluminum to have a certain degree of lithium-ion exchange, and then performing solid-phase exchange (Chinese Patent Application No. 200710121786.2). Hong et al performed ion exchange of a H-SAPO-34 molecular sieve membrane with Li⁺, Na⁺, K⁺, NH₄⁺and Cu²⁺ in a non-aqueous solution, and found that the separation selectivity of CO₂/CH₄ increased by 60%, but CO₂ permeation rate decreased [Micropor. Mesopor. Mater. 106 (2007) 140].

However, in traditional ion exchange methods, a molecular sieve membrane is prepared by dissolving a metal salt in a solvent to form a salt solution, and then placing molecular sieve powders into the membrane in the solution for ion exchange. The ion exchange is slow and the selectivity of the prepared molecular sieve membrane remains to be improved.

### SUMMARY OF THE INVENTION

The technical problem to be solved by the present invention is to provide a method for the pervaporation and vapor-permeation separation of a gas-liquid mixture or a liquid mixture, such as a methanol-containing mixture, by an ion-exchanged SAPO-34 molecular sieve membrane. The present method achieves very high methanol (MeOH) selectivity and permeation flux.

To resolve the issues mentioned above, the present invention provides a method for the pervaporation or vapor-permeation separation of a gas-liquid mixture of a liquid mixture by an ion-exchanged SAPO-34 molecular sieve membrane, said method comprises the following steps:

1) Synthesis of SAPO-34 Molecular Sieve Seeds
mixing and dissolving an Al source, tetraethyl ammonium hydroxide (TEAOH, a template agent), water, a Si source and a P source to make a reaction liquor for seeds, which is then subjected to crystallization for 4-7 h by heating at 170-210° C. (the heating may be microwave heating), then centrifuging, washing and drying to get SAPO-34 molecular sieve seeds;
wherein the molar ratio of the Al source, P source, Si source, tetraethylammonium hydroxide and all water in the reaction liquor for seeds is :1 Al₂O₃: 1-2 P₂O₅: 0.3-0.6 SiO₂: 1-3 TEAOH : 55-150 H₂O.
In this step, the detailed preparation method for the reaction liquor for seeds can be operated as follows:
adding the Al source to the tetraethylammonium hydroxide (TEAOH) solution, and after hydrolysis, secquencially adding the silicon source and the phosphorus source, and then stirring to get the reaction liquor for seeds. More specifically, the operation can be as follows: mixing the tetraethylammonium hydroxide solution with DI water, then adding the Al source to the resultant solution, and stirring for 2-3 h at room temperature. Then adding the Si source dropwise and stirring for 0.5-2 h. Then slowly adding the P source dropwise, stirring for 12-24 h, thereby to get the reaction liquor for seeds.

2) Coating of the Seeds
coating the SAPO-34 molecular sieve seeds onto the inner surface of a porous support to get a porous support coated with SAPO-34 molecular sieve seeds;

3) Synthesis of SAPO-34 Molecular Sieve Membrane
A. Uniformly mixing an Al source, a P source, a Si source, tetraethylammonium hydroxide, di-n-propyl amine (DPA), water and a fluoride to form a mother liquor for SAPO-34 molecular sieve membrane synthesis;
wherein, the molar ratio of the Al source, P source, Si source, tetraethylammonium hydroxide, di-n-propyl amine (DPA) and all water in the mother liquor for molecular sieve membrane synthesis is 1 Al₂O₃: 0.5-3.5 P₂O₅: 0.05-0.6 SiO₂: 0.5-8 TEAOH: 0.1-4.0 DPA: 0.01-1 F⁻: 50-300 H₂O.
In the step A, the peration procedures for forming the mother liquor for molecular sieve membrane synthesis comprises the following steps: mixing the Al source, P source and water, stirring for 1-5 h. Then adding the Si source, stirring for 0.5-2 h; then adding the tetraethylammonium hydroxide, stirring for 0.5-2 h. Then adding di-n-propyl amine, stirring for 0.5-2 h. Then adding the fluoride, stirring for 12-96 h at room temperature to get a homogeneous mother liquor for molecular sieve membrane synthesis.
B. Placing the porous support coated with SAPO-34 molecular sieve seeds prepared in the step 2) in the mother liquor for molecular sieve membrane synthesis and after soaking and aging for 2˜8 h at room temperature-80° C., crystallizing for 3˜24 h at 150˜240° C. to synthesize the SAPO-34 molecular sieve membrane tube.

4) Using the Following Method I or Method II (Two Different Methods for Ion Exchange and Calcination) for Ion Exchange and Calcination to Remove the Template agent:
Method I: supporting a metal salt whose melting point is lower than the calcination temperature (370-700° C.) on the SAPO-34 molecular sieve membrane tube obtained in step 3), drying and then calcining for 2-8 h at 370-700° C., to remove the template agent (tetraethylammonium hydroxide) and simultaneously carry out ion exchange, thereby to obtain an ion-exchanged SAPO-34 molecular sieve membrane;
Method II: calcining the SAPO-34 molecular sieve membrane tube obtained in the step 3) for 2˜8 h at 370-700° C. to remove the template agent (tetraethylammonium hydroxide), then supporting a metal salt whose melting point is lower than the calcination temperature (370-700° C.) on the molecular sieve membrane tube having the template agent removed, and drying, then ion-exchanging in melt state at a temperature lower than the calcination temperature of 300-700° C. and higher than the melting point of the metal salt, thereby to obtain an ion-exchanged SAPO-34 molecular sieve membrane.

5) Using the ion-exchanged SAPO-34 molecular sieve membrane obtained in step 4) to perform separation of a gas-liquid mixture or a liquid mixture by a process of pervaporation separation or vapor-permeation separation. The gas in the gas-liquid mixture is selected from common gases, for example includes inert gas, hydrogen gas, oxygen gas, CO₂ or gaseous hydrocarbon, and the liquid in the gas-liquid mixture is selected from common solvents such as water, alcohol, ketone or aromatics;
Wherein in the step 5), the inert gas contains N₂;
the gaseous hydrocarbon contains methane;
the alcohol contains methanol, ethanol, or propanol;
the ketone contains acetone or butanone;
the aromatics contain benzene.

In addition, in the step 5), in the separation of the liquid mixture by the ion-exchanged SAPO-34 molecular sieve membrane, said liquid mixture is a mixture of methanol and a liquid other than methanol, said liquid other than methanol is selected from one of dimethyl carbonate, ethanol, methyl tert-butyl ether.

In the steps 1) and 3), the Al source is selected from one or more of aluminum isopropoxide, Al(OH)₃, elemental aluminum, an Al salt; wherein, said Al salt is selected from one or more of aluminum nitrate, aluminum chloride, aluminum sulfate, and aluminum phosphate.

In the steps 1) and 3), the P source is phosphoric acid; the Si source is selected from one or more of tetraethyl orthosilicate, tetramethyl orthosilicate, silica sol, silica, sodium silicate, water glass.

In the step 1), the heating is preferably microwave heating; the size of the SAPO-34 molecular sieve seeds is 50-1000 nm.

In the step 2), the porous support is selected from a porous ceramic tube, wherein the pore size of the porous ceramic tube is 5-2000 nm, and the material of the porous ceramic tube includes Al₂O₃, TiO_{2,} ZrO_{2,} SiC or silicon nitride.

The coating of the seeds in the step 2) comprises the following steps: sealing the two ends of the porous support tube with glaze, washing and drying, sealing the outer surface, and then coating the SAPO-34 molecular sieve seeds onto the inner surface of the porous support; the coating method is selected from brush coating or dip coating.

In the step 3), the fluoride is selected from one or a mixture of HF and a fluoride salt; wherein the fluoride salt is selected from a fluoride salt of a main-group metal and a fluoride salt of a transition metal. For example, the fluoride salt is selected from potassium fluoride, sodium fluoride, or ammonium fluoride.

In the step 4), the cation of the metal salt is a main-group metal or a transition metal, the anion is a hydracid radical or an oxo acid radical. Typical metal salt is selected from sodium nitrate, lithium nitrate, rubidium nitrate, magnesium nitrate, potassium nitrate, sodium chlorate, or sodium perchlorate.

In the step 4), in the Method I or Method II, the method of supporting the metal salt whose melting point is lower than the calcination temperature includes supporting the metal salt on the front surface, back surface or both (preferably front surface) of the molecular sieve membrane tube by dip coating, spin coating, spray coating or brush coating. The operation procedures of supporting the metal salt by dip coating comprises the following steps: in the Method I or Method II, placing the molecular sieve membrane having or not having the template agent removed in a 0.01˜50 wt % (preferably 0.1˜50 wt %) solution of the metal salt and soaking for 1 s-2 days (preferably 1 s-180 min) at −40-100° C.; the solvent in the solution of the metal salt is selected from water, acetone, or alcohol.

In the step 4), the drying temperature ranges from room temperature to 200° C.; the conditions for ion exchanging in melt state are: that the ion exchange temperature is 100˜500° C. and the ion exchange time is 1˜8 h.

In the step 4), the atmosphere for calcination is selected from: inert gas, vacuum, air, oxygen gas, or diluted oxygen in any ratio; in the calcination, the temperature increasing rate and the temperature decreasing rate are not higher than 2K/min.

In the step 5), the conditions for the process of pervaporation separation or vapor-permeation separation are: methanol concentration in the feed: 1-99 wt %, feed flow rate: 1˜500 mL/min, separation operation temperature: room temperature˜150° C., pressure on the permeate side: 0.06-300 Pa.

This invention prepared an ion-exchanged SAPO-34 molecular sieve membrane on a porous support and used the prepared ion-exchanged SAPO-34 molecular sieve membranes to perform pervaporation/vapor-permeation separation of a gas-liquid mixture and a liquid mixture, e.g. methanol/dimethyl carbonate (methanol/DMC) mixture. The molecular sieve membrane has very high MeOH selectivity and permeation flux. For example, at an operation temperature of room temperature to 150° C., the separation factor for separating a methanol/dimethyl carbonate (70/30) azeotrope is above 2000, and the methanol content in the permeate is above 99.99 wt %. Thus, the present invention provides a high efficiency, environmental friendly and economic method for separation of methanol/dimethyl carbonate. The present method for membrane separation of methanol-dimethyl carbonate has advantages like low energy consumption, being not limited by azeotropic mixture, high methanol flux and high separation factors and thus has great economic value.

Besides the separation of methanol/dimethyl carbonate mixture (methanol/dimethyl carbonate azeotrope), the ion-exchanged SAPO-34 molecular sieve membrane of the present invention could also be used for the pervaporation or vapor-permeation separation of a mixture of methanol and other liquid, such as methanol-ethanol, methanol-methyl tert-butyl ether.

In addition, the ion-exchanged SAPO-34 molecular sieve membrane of the present invention can also be used for the pervaporation or vapor-permeation separation of a gas-liquid mixture.

### BRIEF DESCRIPTION OF THE DRAWINGS

The invention will be explained in further detail by taking the following figures and the detailed implementation.

FIG. 1 is a SEM (Scanning Electron Microscopy) image of SAPO-34 seeds of Example 1;

FIG. 2 is an XRD (X-ray diffraction) pattern of SAPO-34 seeds of Example 1;

FIG. 3 is a SEM image of SAPO-34 molecular sieve membrane prepared in Example 1 (a potassium ion-exchanged molecular sieve membrane obtained by simultaneous ion exchange and removal of the template agent); wherein, FIG. 3A is a surface SEM image of the ion-exchanged SAPO-34 molecular sieve membrane; FIG. 3B is a cross sectional SEM image of the ion-exchanged SAPO-34 molecular sieve membrane;

FIG. 4 is a SEM image of un-exchanged SAPO-34 molecular sieve membrane in Example 1; wherein, FIG. 4A is a surface SEM image of the un-exchanged SAPO-34 molecular sieve membrane; FIG. 4B is a cross sectional SEM image of the un-exchanged SAPO-34 molecular sieve membrane;

FIG. 5 is a schematic diagram of a pervaporation separation process, wherein 1 denotes feed liquid, 2 denotes peristaltic pump, 3 denotes molecular sieve membrane assembly and heat source, 4 denotes stop valve, 5 denotes cold trap, 6 denotes vacuum gauge, 7 denotes vacuum pump;

FIG. 6 is surface SEM image of SAPO-34 molecular sieve membrane of Example 3 (a sodium ion-exchanged molecular sieve membrane obtained by removal of the template agent followed by ion exchange in melt state);

FIG. 7 is a cross sectional SEM image of SAPO-34 molecular sieve membrane of Example 3 (a sodium ion-exchanged molecular sieve membrane obtained by removal of the template agent followed by ion exchange in melt state).

### EXAMPLES

### Example 1: Separation of Methanol/Dimethyl Carbonate by a Potassium Ion-Exchanged SAPO-34 Molecular Sieve Membrane Obtained by Simultaneous Ion Exchange and Removal of Template Agent

Step1: 2.46 g of DI water were added to 31.13 g of tetraethyl ammonium hydroxide solution (TEAOH, 35 wt %) . Then 7.56 g of aluminum isopropoxide were added thereto, and the resultant was stirred for 2-3 h at room temperature; then 1.665 g of silica sol (40 wt %) was added dropwise, and the resultant was stirred for 1 h. Finally, 8.53 g of phosphoric acid solution (H₃PO₄, 85 wt %) were slowly added dropwise, and the resultant was stirred overnight (e.g., stirred for 12 h). Then crystallization was performed at 180° C. for 7 h by using microwave heating. The obtained product was taken out from the reactor, centrifuged, washed and dried to obtain SAPO-34 molecular sieve seeds.

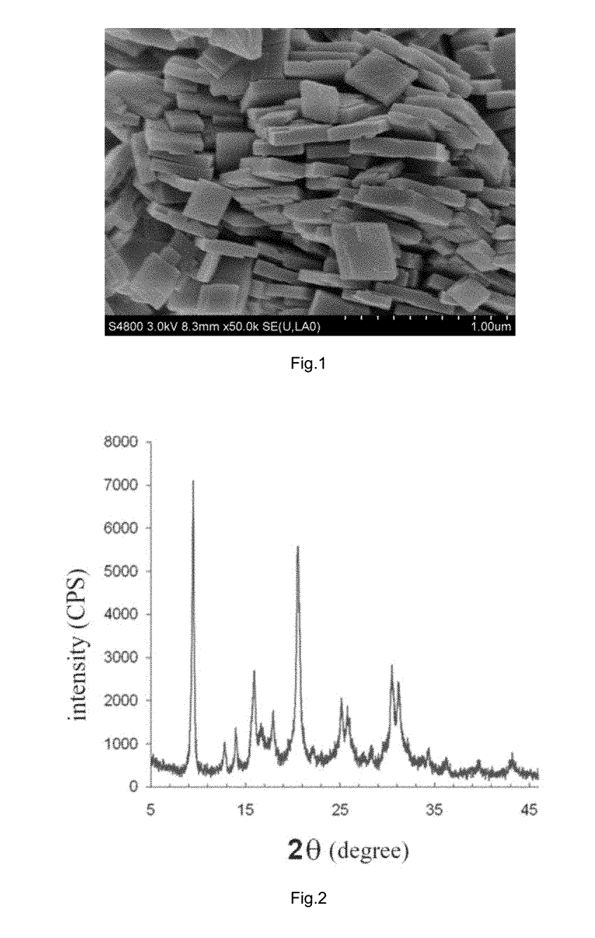

The SEM image and XRD pattern of the seeds are shown in FIG. 1 and FIG. 2, respectively. It can be seen from the SEM image that the size of the seeds is around 300 nm * 300 nm * 100 nm. The XRD pattern indicates that the seeds are pure SAPO-34 phase, and are well crystallized with no impure phase.

Step 2: A porous ceramic tube (material: alumina) with 5 nm pore size was used as a support. The two ends of the support were sealed with glaze. After washing and drying, the out surface of the support was sealed (covered) by PTFE tape. Then the SAPO-34 molecular sieve seeds were coated onto the inner surface of the ceramic tube by brush coating method.

Step 3: 4.27 g of phosphoric acid solution (H₃PO₄, 85 wt %) were mixed with 43.8 g of DI water, and the resultant was stirred for 5 min. Then 7.56 g of aluminum isopropoxide were added, and the resultant was stirred for 3 h at room temperature. 0.83 g of silica sol (40 wt %) were added, and the resultant was stirred for 30 min at room temperature. Then, 7.78 g of tetraethyl ammonium hydroxide solution (TEAOH, 35 wt %) were added dropwise, and the resultant was stirred for 1 h at room temperature. Finally, 3.0 g of di-n-propylamine were added, and the resultant was stirred for 30 min at room temperature, then 0.045 g of hydrofluoric acid (HF, 40 wt %) were added, and the resultant was stirred overnight (e.g., stirred for 12 hours) at 50° C., getting a mother liquor for synthesis of SAPO-34 molecular sieve membrane. The porous ceramic tube coated with SAPO-34 molecular sieve seeds, which was prepared in the above step 2, was placed in a reaction vessel, and the mother liquor for synthesis of molecular sieve membrane was added. The reaction vessel was closed and aging was performed for 3 h at room temperature. Then hydrothermal synthesis was performed at 220° C. for 5 h. After taken out from the reaction vessel, the product was thoroughly rinsed and dried in an oven.

Step 4: The membrane tube obtained in step 3 was placed in a 1 wt % potassium nitrate aqueous solution and soaked for 3 min, then taken out and dried at room temperature. Then the membrane tube was calcined in vacuum at 400° C. for 4 h to remove the template agent (the temperature increasing rate and temperature decreasing rates were 1° C./min, respectively), getting an ion-exchanged SAPO-34 molecular sieve membrane.

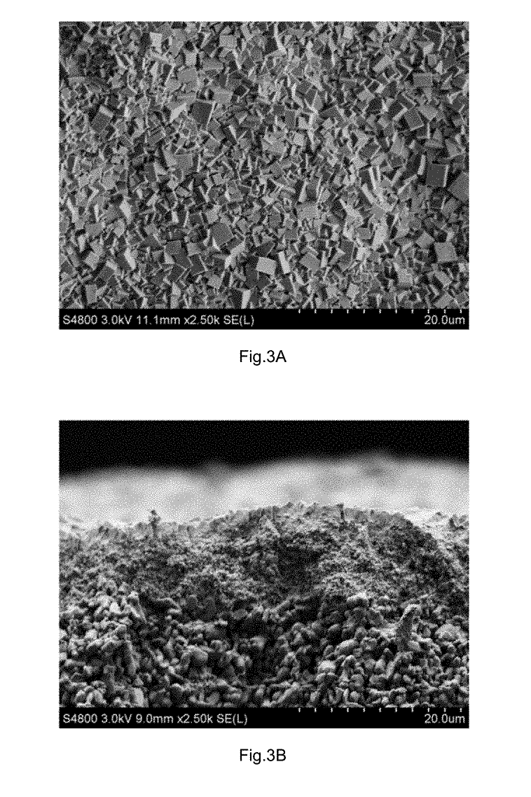

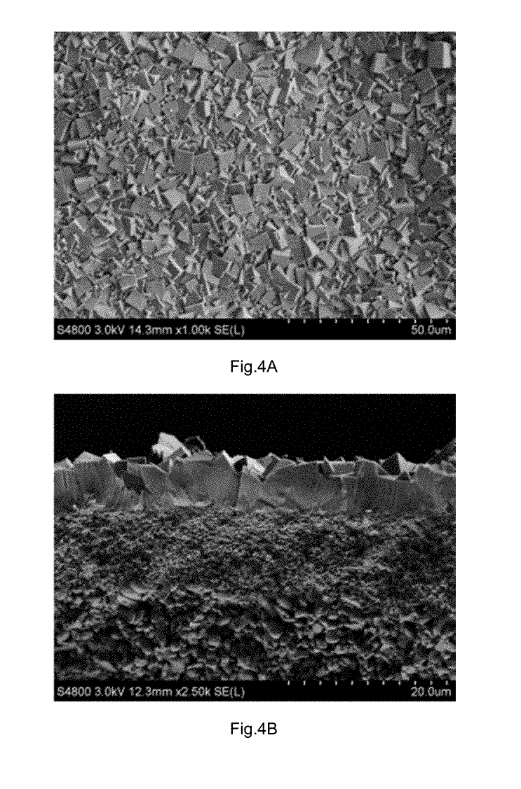

The surface and cross sectional SEM images of the ion-exchanged SAPO-34 molecular sieve membrane are respectively shown in FIGS. 3A and 3B. The surface and cross sectional SEM images of an un-exchanged SAPO-34 molecular sieve membrane prepared under the same conditions are respectively shown in FIGS. 4A and 4B. It can be seen from the SEM images in FIGS. 3 and 4 that their support surfaces are both completely covered by square lamellar SAPO-34 crystals which are perfectly cross-linked therebetween. The crystal size is 4-7 microns, and the molecular sieve membrane surface is flat. The cross sectional image shows that the thickness of the membrane is about 5-6 microns. Thus, ion exchange has no significant effect on the morphology of membrane.

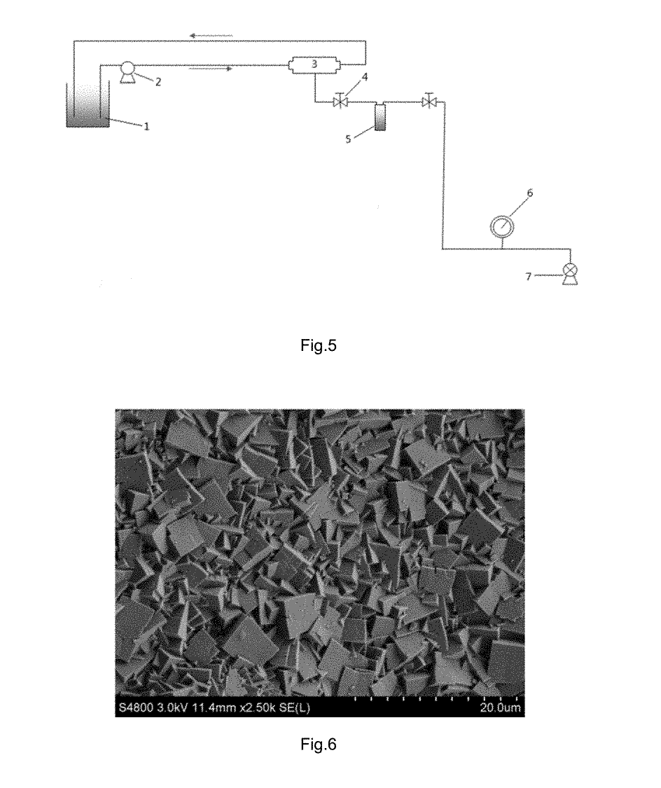

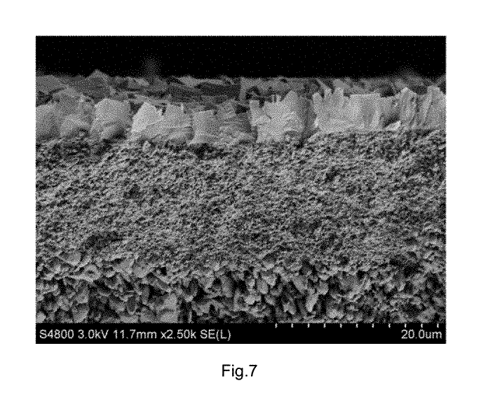

Step 5. The ion-exchanged SAPO-34 molecular sieve membrane obtained in the above step was used to separate a methanol/dimethyl carbonate (i.e., DMC/MeOH) azeotrope by a pervaporation process, wherein the feed flow rate was 1 mL/min, the separation operation temperature 70° C., the pressure on the permeate side 100 Pa and the composition of the MeOH/DMC feed was from 90/10 to 70/30 (mass ratio). The schematic diagram of the pervaporation process is shown in FIG. 5.

The separation factor is calculated from: α=(w₂ₘ/w_{2d})/(w₁ₘ/w_{1d}), where w₂ₘ is the mass concentration of methanol on the permeate side, w_{2d} is the mass concentration of dimethyl carbonate on the permeate side, w₁ₘ is the mass concentration of methanol in the feed and w_{1d} is the mass concentration of dimethyl carbonate in the feed.

The permeation flux equation is J=Δm/(s×t), wherein Δm is the mass (g) of a product collected on the permeate side, s is the molecular sieve membrane area (m²) and t is the collecting time (h).

It can be seen from Table 1 that when the feed composition of MeOH/DMC is from 90/10 to 70/30, the methanol selectivity of the SAPO-34 membrane is more than 2000, and the flux is about 0.14 kg/(m²·h) (Table 1). Thus, the ion-exchanged SAPO-34 molecular sieve membrane has a very high methanol-dimethyl carbonate separation factor.

TABLE 1
The pervaporation separation test results of MeOH/DMC in Example 1.

| Feed composition | Permeation flux J | Separation |
|---|---|---|
| MeOH/DMC (wt %) | [kg/(m² · h)] | factor α |
| 70/30 | 0.137 | 2500 |
| 90/10 | 0.146 | 2000 |

### Example 2: Separation of Methanol/Dimethyl Carbonate Mixture at 120° C. by Ion-Exchanged SAPO-34 Membrane.

All steps in this Example are the same as in Example 1 except that the feed composition of MeOH/DMC is 90/10 (mass ratio), the separation operation temperature is 120° C., and the permeate side pressure is 0.3 Mpa in step 5.

TABLE 2
The vapor permeation separation test results of MeOH/DMC
in Example 2.

| Operation | | |
|---|---|---|
| temperature | Permeation flux J | Separation |
| ° C. | [kg/(m² · h)] | factor α |
| 120 | 2.3 | 4100 |

It can be seen from Table 2 that when the feed composition of MeOH/DMC is 90/10, and the operating temperature is 120° C., the methanol selectivity of the ion exchanged SAPO-34 molecular sieve membrane is greater than 4000, and the flux is greatly increased compared to that at 70° C. The increase of the flux is due to the fact that the increase of feed pressure causes the increasing of mass transfer driving force of methanol. Thus it can be seen that the ion-exchanged SAPO-34 molecular sieve membrane has a very high methanol-dimethyl carbonate separation factor and a high methanol flux.

### Example 3: Separation of Methanol/Dimethyl Carbonate by SAPO-34 Molecular Sieve Membrane Prepared by Removal of Template Agent Followed by Sodium Ion EXchange in Melt State

All the steps in this Example are the same as in Example 1, except that in step 4, the molecular sieve membrane tube obtained in step 3 was calcined in vacuum at 400° C. for 4 h to remove the template agent, cooled down to room temperature, and then placed in a 1 wt % sodium nitrate aqueous solution and soaked for 3 min, then taken out and dried at room temperature; then calcined at 310° C. for 8 h to carry out ion exchange, thereby to get a sodium ion-exchanged molecular sieve membrane. In step 5, the feed composition of MeOH/DMC is 90/10 (mass ratio), the separation operation temperature is 120° C., and the pressure on the permeate side is 0.3 MPa.

TABLE 3
The vapor permeation separation test results of MeOH/DMC
in Example 3.

| Permeate | | |
|---|---|---|
| side | | |
| pressure | Permeation flux J | Separation |
| MPa | [kg/(m² · h)] | factor α |
| 0.3 | 2.1 | 3700 |

It can be seen from Table 3 that when the feed composition of MeOH/DMC is 90/10, and the operating temperature is 120° C., the methanol selectivity of the SAPO-34 molecular sieve membrane which is sodium ion-exchanged in melt state is greater than 3500, and the permeation flux is greater than 2 kg/(m².h). Thus it can be seen that the ion-exchanged SAPO-

[symbol]

1. A method for the separation of a gas-liquid mixture or a liquid mixture by preparing and using an ion-exchanged SAPO-34 molecular sieve membrane, said method comprises the following steps:
1) mixing and dissolving an Al source, tetraethyl ammonium hydroxide (TEAOH), water, a Si source and a P source to make reaction liquor for seeds, which is then subjected to crystallization for 4-7 h by heating at 170-210° C., then centrifuging, washing and drying to get SAPO-34 molecular sieve seeds; wherein the molar ratio of the Al source, P source, Si source, tetraethylammonium hydroxide and all water in the reaction liquor for seeds is :1 Al₂O₃: 1-2 P₂O₅: 0.3-0.6 SiO₂: 1-3 TEAOH : 55-150 H₂O;
2) coating the SAPO-34 molecular sieve seeds onto the inner surface of a porous support to get a porous support coated with SAPO-34 molecular sieve seeds;
3) synthesis of SAPO-34 molecular sieve membrane tube,
A. uniformly mixing an Al source, a P source, a Si source, tetraethylammonium hydroxide, di-n-propyl amine (DPA), water and a fluoride to form a mother liquor for SAPO-34 molecular sieve membrane synthesis;
wherein, the molar ratio of the Al source, P source, Si source, tetraethylammonium hydroxide, di-n-propyl amine (DPA) and all water in the mother liquor for molecular sieve membrane synthesis is 1 Al₂O₃ : 0.5-3.5 P₂O₅: 0.05-0.6 SiO₂: 0.5-8 TEAOH : 0.1-4.0 DPA : 0.01-1 F: 50-300 H₂O;
B. placing the porous support coated with SAPO-34 molecular sieve seeds prepared in the step 2) in the mother liquor for molecular sieve membrane synthesis and after soaking and aging for 2˜8 h at room temperature˜80° C., crystallizing for 3-24 h at 150˜240° C. to synthesize the SAPO-34 molecular sieve membrane tube;
4) using the following Method I or Method II for ion exchange and calcination,
Method I: supporting a metal salt whose melting point is lower than 370-700° C. on the SAPO-34 molecular sieve membrane tube obtained in step 3), drying and then calcining for 2˜8 h at 370-700° C., to remove the template agent tetraethylammonium hydroxide and simultaneously carry out ion exchange, thereby to obtain an ion-exchanged SAPO-34 molecular sieve membrane;
Method II: calcining the SAPO-34 molecular sieve membrane tube obtained in the step 3) for 2-8 h at 370-700° C. to remove the template agent tetraethylammonium hydroxide, then supporting a metal salt whose melting point is lower than 370-700° C. on the molecular sieve membrane tube, and drying, then ion-exchanging in melt state at a temperature lower than the calcination temperature of 370-700° C. and higher than the melting point of the metal salt, thereby to obtain an ion-exchanged SAPO-34 molecular sieve membrane;
wherein in Method I and MethodII the method of supporting the metal salt whose melting point is lower than the calcination temperature is performed by supporting the metal salt on the front surface, back surface or both of the molecular sieve membrane tube by dip coating, spin coating, spray coating or brush coating:
5) using the ion-exchanged SAPO-34 molecular sieve membrane obtained in step 4) to perform separation of a gas-liquid mixture by a process of vapor-permeation separation, wherein, the gas in the gas-liquid mixture is selected from inert gas, hydrogen gas, oxygen gas, CO₂ or gaseous hydrocarbon, and the liquid in the gas-liquid mixture is selected from alcohol, ketone or aromatics; or
using the ion-exchanged SAPO-34 molecular sieve membrane obtained in step 4) to perform separation of a liquid mixture by a process of pervaporation separation, wherein said liquid mixture is a mixture of methanol and a liquid other than methanol, said liquid other than methanol is selected from one of dimethyl carbonate, ethanol, methyl tert-butyl ether.

2. The method according to claim 1, characterized in that in the steps 1) and 3), the Al source is selected from one or more of aluminum isopropoxide, Al(OH)₃, elemental aluminum, an Al salt; wherein said Al salt is selected from one or more of aluminum nitrate, aluminum chloride, aluminum sulfate, and aluminum phosphate; , the P source is phosphoric acid; the Si source is selected from one or more of tetraethyl orthosilicate, tetramethyl orthosilicate , silica sol, silica, sodium silicate, water glass.

3. The method according to claim 1, characterized in that in the step 1), the heating is microwave heating and the size of the SAPO-34 molecular sieve seeds is 50˜1000 nm.

4. The method according to claim 1, characterized in that in the step 2), the porous support is a porous ceramic tube; wherein the pore size of the porous ceramic tube is 5-2000 nm, and the material of the porous ceramic tube includes Al₂OP₃, TiO₂, ZrO₂, SiC or silicon nitride.

5. The method according to claim 1, characterized in that the coating of the seeds in step 2), the process comprises sealing the two ends of the porous support with glaze, washing and drying, sealing the outer surface, and then coating the SAPO-34 molecular sieve seeds onto the inner surface of the porous support; and the coating is selected from brush coating or dip coating.

6. The method according to claim 1, characterized in that in the step 3), the fluoride is selected from one or a mixture of HF and a fluoride salt; wherein the fluoride salt is selected from a fluoride salt of a main-group metal and a fluoride salt of a transitional metal.

7. The method according to claim 6, characterized in that the fluoride is selected from potassium fluoride, sodium fluoride, or ammonium fluoride.

8. The method according to claim 1, characterized in that in the step 3), the operation procedures for forming the mother liquor for molecular sieve membrane synthesis comprises the following steps
mixing the Al source, P source and water, stirring for 1˜5 h; then adding the Si source, stirring for 0.5˜2 h; then adding the tetraethylammonium hydroxide, stirring for 0.5˜2 h; then adding di-n-propyl amine, stirring for 0.5˜2 h; then adding the fluoride, stirring for 12˜96 h at room temperature to get a homogeneous mother liquor for molecular sieve membrane synthesis.

9. The method according to claim 1, characterized in that in the step 4), the cation of the metal salt is a main-group metal or a transitional metal, and the anion is a hydracid radical or an oxo acid radical.

10. The method according to claim 9, characterized in that the metal salt is selected from sodium nitrate, lithium nitrate, rubidium nitrate, magnesium nitrate, potassium nitrate, sodium chlorate, or sodium perchlorate.

11. The method according to claim 14, characterize in that the method of supporting the metal salt whose melting point is lower than the calcination temperature is supporting the metal salt by dip coating, the operation procedure thereof comprises the following steps,
in the Method I or Method II, the molecular sieve membrane having or not having the template agent removed is placed in a 0.01˜50 wt % solution of the metal salt and soaked for 1 s˜2 days at −40˜100° C.; wherein the solvent in the solution of the metal salt is selected from water, acetone, or alcohol.

12. The method according to claim 11, characterize in that in the ion exchange in Method I or Method II, the molecular sieve membrane having or not having the template agent removed is placed in a 0.1˜50 wt % solution of the metal salt and soaked for 1 s˜180 min at −40˜100° C.

13. The method according to claim 1, characterize in that in the step 4), the drying temperature ranges from room temperature to 200° C.; the conditions for ion exchanging in melt state are that the ion exchange temperature is 100˜500° C. and the ion exchange time is 1˜8 h; wherein), the atmosphere for calcination is selected from inert gas, vacuum, air, oxygen, or diluted oxygen in any ratio; and in the calcination, the temperature increasing rate and the temperature decreasing rate are not higher than 2 K/min.

14. The method according to claim 14, characterize in that in step 5), the conditions for the process of pervaporation separation are that the methanol concentration in the feed is 1˜99 wt %, the feed flow rate is 1˜500 mL/min, the separation operation temperature is room temperature˜150° C., pressure on the permeate side is 0.06˜300 Pa.
